(19) United States
(12) Patent Application Publication
(10) Pub. No.: US 2026/0315025 A1
(43) Pub. Date: Oct. 15, 2026

(54) DYNAMIC IMAGE ANALYSIS APPARATUS, DYNAMIC IMAGE PROCESSING SYSTEM, AND NON-TRANSITORY COMPUTER-READABLE STORAGE MEDIUM STORING EXTUBATION DETERMINATION INFORMATION PROVIDING PROGRAM

(71) Applicant: Konica Minolta, Inc., Tokyo (JP)

(72) Inventors: Kojiro Ono, Chiba (JP); Kentaro Okamoto, Tokyo (JP); Ryoichi Watanabe, Tokyo (JP)

(73) Assignee: Konica Minolta, Inc., Tokyo (JP)

(21) Appl. No.: 19/644,046

(22) Filed: Apr. 10, 2026

(30) Foreign Application Priority Data
Apr. 11, 2025 (JP) 2025-065670

(51) Int. Cl. A61B 6/00; A61B 6/50; A61M 16/00; G06T 7/00; G16H 50/30

(52) U.S. Cl. CPC A61B 6/5217; A61B 6/4405; A61B 6/507; A61M 16/0003; G06T 7/0012; G16H 50/30; G06T 2207/10116; G06T 2207/30048; G06T 2207/30061

(57) ABSTRACT

A dynamic image analysis apparatus, includes: one or more hardware processors; in which the one or more hardware processors are configured to: collectively acquire respiratory information related to respiration and heartbeat information related to heartbeat, which are information allowing evaluation of eligibility for extubation of a ventilator, from a dynamic image captured by irradiating a chest with radiation while a subject with a ventilator attached thereto is in a quiet breathing state; and output the respiratory information and the heartbeat information as a list.

### CROSS REFERENCE TO RELATED APPLICATIONS

The entire disclosure of Japanese Patent Application No. 2025-065670 filed on Apr. 11, 2025 is incorporated herein by reference in its entirety.

### BACKGROUND

### Technological Field

The present invention relates to a dynamic image analysis apparatus, a dynamic image processing system, and a non-transitory computer-readable storage medium storing an extubation determination information providing program.

### Description of Related Art

Among patients who enter an Intensive Care Unit (ICU) or the like, not a few patients need a ventilator with tracheal intubation. For a patient wearing a ventilator, long-term tracheal intubation is accompanied by a risk of occurrence of respiratory complications, and therefore, early weaning from mechanical ventilation (extubation) is desirable whenever possible. Currently, determination of extubation is performed using criteria for extubation such as the Spontaneous Breathing Trial (SBT), for example. However, the SBT has a large number of determination items and is accompanied by subjective judgment, so that the judgment is sometimes difficult for a doctor and the psychological burden is also large.

Japanese Unexamined Patent Publication No. 2018-130264 proposes an invention for objectively evaluating a respiratory state of a patient during wearing of a ventilator or during extubation, and indicates that evaluation information for evaluating the respiratory state is generated and displayed from a dynamic image which is captured using radiation and indicates a dynamic state of a thorax of the patient.

In Japanese Unexamined Patent Publication No. 2018-130264, information related to respiration and heartbeats such as a respiratory rate and a heart rate is measured separately by attaching a respiration sensor and a pulse oximeter to a patient. Therefore, the evaluation information for evaluating the respiratory state and the information on the respiration and the heartbeat are not information synchronized with each other, a correspondence relation therebetween is not clear, and the information is not necessarily highly reliable information in determining whether to remove the tube. In addition, since the evaluation information for evaluating the respiratory state and the information relating to the respiration and the heartbeat are separately acquired information, it is necessary to confirm the information in each device, which takes time and effort, and it takes time to make a determination.

In view of the above, in the SBT having a large number of determination items, it is desirable that information useful for determination of extubation can be collectively confirmed.

### SUMMARY

Objectives of the present invention include providing a dynamic image analysis apparatus, a dynamic image processing system, and a non-transitory computer-readable storage medium storing an extubation determination information providing program, which are capable of collectively acquiring information useful for determination of extubation from a ventilator and providing the information in the form of a list.

In order to achieve at least one of the above-described objectives, a dynamic image analysis apparatus reflecting one aspect of the present invention includes:
an acquisition section configured to collectively acquire respiratory information related to respiration and heartbeat information related to heartbeat, which are information allowing evaluation of eligibility for extubation of a ventilator, from a dynamic image captured by irradiating a chest with radiation while a subject with a ventilator attached thereto is in a quiet breathing state; and
an output section that outputs the respiratory information and the heartbeat information as a list.

In order to achieve at least one of the above-described objectives, a dynamic image processing system reflecting one aspect of the present invention includes:
a medical cart including a dynamic imaging apparatus for capturing a dynamic image by irradiating a chest with radiation while a subject with a ventilator attached thereto is in a quiet breathing state, and a cart in which the dynamic imaging apparatus is mounted and that is configured to be movable; and
the dynamic image analysis apparatus described above.

In order to achieve at least one of the above-mentioned objectives, a non-transitory computer-readable storage medium storing an extubation determination information providing program reflecting one aspect of the present invention causes execution of:
a process of collectively acquiring respiratory information related to respiration and heartbeat information related to heartbeat, which are information allowing evaluation of eligibility for extubation of a ventilator, from a dynamic image captured by irradiating a chest with radiation while a subject with a ventilator attached thereto is in a quiet breathing state; and
a process of outputting the respiratory information and the heartbeat information as a list.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating an example of an overall configuration of a dynamic image processing system;

FIG. 2 is a block diagram illustrating a functional configuration of an apparatus main body configuring the dynamic imaging apparatus;

FIG. 3 is a block diagram illustrating an example of a functional configuration of the dynamic image analysis apparatus;

FIG. 4 is a diagram illustrating SBT initiation safety criteria and SBT success criteria in a ventilator weaning protocol;

FIG. 5 is a flowchart illustrating a diagnostic index providing method performed by the dynamic image analysis apparatus;

FIG. 6 is a diagram illustrating a method of obtaining the respiratory rate per minute and a diaphragm displacement amount from a dynamic image captured by a dynamic imaging apparatus;

FIG. 7 is a diagram showing an analysis summary including the respiratory rate per minute, the diaphragm displacement amount, and the diagnostic index obtained by the method described in FIG. 6;

FIG. 8 is a flowchart illustrating another diagnostic index providing method performed by the dynamic image analysis apparatus;

FIG. 9 is a diagram illustrating another method of obtaining the respiratory rate per minute and the diaphragm displacement amount from the dynamic image captured by the dynamic imaging apparatus;

FIG. 10 is a diagram showing an analysis summary including the respiratory rate per minute, the diaphragm displacement amount, and the diagnostic index obtained by the method described in FIG. 9; and

FIG. 11 is a diagram illustrating an analysis summary including respiratory information and heartbeat information obtained from dynamic images captured by the dynamic imaging apparatus.

### DETAILED DESCRIPTION OF EMBODIMENTS

Hereinafter, one or more embodiments of the present invention will be described with reference to the drawings. However, the scope of the invention is not limited to the disclosed embodiments.

Hereinafter, embodiments of the present invention will be described in detail with reference to the drawings.

### Dynamic Image Processing System

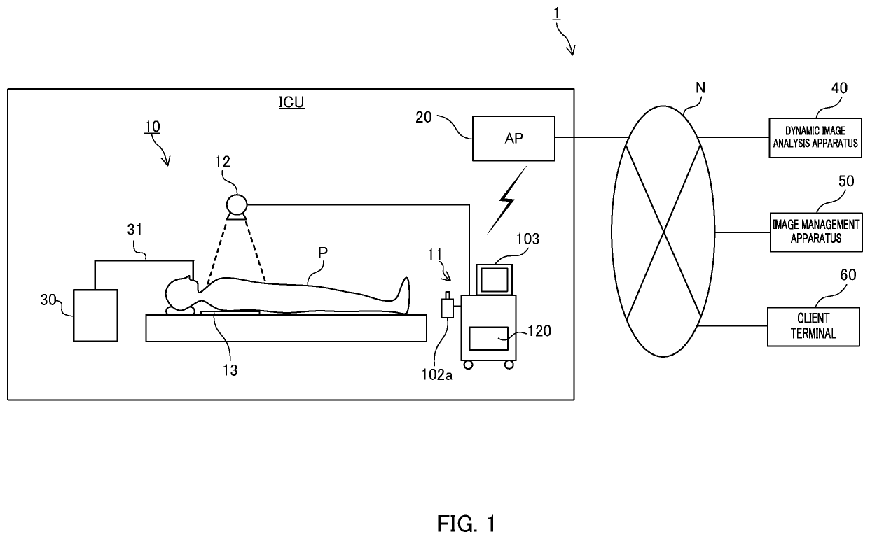

FIG. 1 is a view illustrating an example of an overall configuration of a dynamic image processing system 1.

The dynamic image processing system 1 includes at least a dynamic imaging apparatus 10 and a dynamic image analysis apparatus 40. The dynamic image processing system 1 may further include an image management apparatus 50 and a client terminal 60.

The dynamic imaging apparatus 10 is configured as a medical cart in one example, and is a movable apparatus as described below, which is suitable for visiting a patient P (subject) who has difficulty in moving to another room. The dynamic imaging apparatus 10 performs dynamic imaging for capturing a dynamic image of the patient P, and is suitable for dynamic imaging of the patient P who enters an ICU and is difficult to move to another room, for example, as illustrated in FIG. 1.

Here, the dynamic imaging refers to obtaining a plurality of frame images by repeatedly irradiating the patient P with pulsed radiation (for example, X-rays) at a predetermined frame rate (pulse irradiation). The dynamic image refers to a series of frame images obtained by dynamic imaging. Dynamic analysis described later refers to analysis processing performed on a dynamic image and includes processing of analyzing the dynamic image to emphasize or attenuate (remove) a predetermined structure in addition to processing of analyzing the movement of a subject based on the dynamic image.

The dynamic images captured by the dynamic imaging apparatus 10 are transmitted to the dynamic image analysis apparatus 40. The dynamic image analysis apparatus 40 performs dynamic analysis on the dynamic images transmitted from the dynamic imaging apparatus 10. Details of the dynamic image analysis apparatus 40 will be described later.

The dynamic images captured by the dynamic imaging apparatus 10 and an analysis result of the dynamic analysis performed by the dynamic image analysis apparatus 40 are transmitted to the image management apparatus 50. The image management apparatus 50 manages the dynamic images and the analysis result obtained by dynamically analyzing the dynamic images in association with each other. The image management apparatus 50 is a medical image management system, for example, a Picture Archiving and Communication System (PACS).

The client terminal 60 is a terminal used by a healthcare professional such as a doctor, and reads the dynamic images and the analysis result from the image management apparatus 50 and presents them so that the healthcare professional can view them, based on the operation of the healthcare professional.

### Dynamic Image Capturing Apparatus

The dynamic imaging apparatus 10 includes an apparatus main body 11, a radiation source 12, and a Flat Panel Detector (FPD) 13.

The apparatus main body 11 is an apparatus that performs dynamic imaging of the dynamic images using radiation, which will be described in detail later. The apparatus main body 11 is mounted on a cart configured to be movable by wheels or the like, and is configured as a medical cart that is movable to the vicinity of the patient P. Note that the dynamic imaging apparatus 10 may be portable, and in that case, may not include wheels. The dynamic imaging apparatus 10 may be arranged in a photographing room, and in that case, a movable patient P is a photographing target.

In addition, the apparatus main body 11 is connected to a network N such as an in-hospital Local Area Network (LAN) via an Access Point (AP) 20 installed in a hospital by communication, for example, wireless communication. The apparatus main body 11 can transmit and receive data to and from another apparatus via the network N. The other apparatuses are the dynamic image analysis apparatus 40, the image management apparatus 50, the client terminal 60, and the like described above.

The radiation source 12 irradiates an imaging target site of the patient P with radiation (X-rays) under the control of the apparatus main body 11. In the case of dynamic imaging, the radiation source 12 directs, for example, pulsed radiation to the patient P. Although not shown, the radiation source 12 is attached to, for example, an arm of the apparatus main body 11. The arm is configured to be movable so that the radiation source 12 can be placed facing the imaging target site of the patient P.

The FPD 13 is a portable radiation detector corresponding to dynamic imaging, and detects a radiation transmitted through the imaging target site of the patient P. Various known FPDs can be used as the FPD 13. In addition, the irradiation with the radiation by the radiation source 12 and the radiography by the FPD 13 are configured to be timed with each other by known synchronization control, for example, time correction by time synchronization communication.

The FPD 13 includes, for example, radiation detection elements two dimensionally arranged on a glass substrate. The radiation detection elements are formed by semiconductor image sensors, such as photodiodes. The radiation detection elements detect, in accordance with the intensity of radiation, radiation that has been irradiated from the radiation source 12 and has passed through the subject P, convert the detected radiation into electrical signals, and accumulate the electrical signals. Each of the radiation detection elements is connected to a switching section, for example, a Thin Film Transistor (TFT) or the like, and accumulation and reading of the electric signals are controlled by the switching section so as to be output as image data.

The patient P is a patient to whom the ventilator 30 is attached. The ventilator 30 is an example of a respiratory assistance device.

Here, the ventilator 30 will be briefly described. The ventilator 30 is a device that performs mechanical ventilation and artificial respiration by taking respiratory gas into and out of the lungs in order to assist the respiration of the patient P who cannot breathe or does not breathe well. There are several methods for attaching the ventilator 30, but for the patient P in the ICU, for example, tracheal intubation in which the tube 31 is inserted into the trachea from the mouth or the nose, tracheotomy in which the trachea is incised and the tube 31 is inserted into the trachea, or the like is used.

In addition, the dynamic imaging apparatus 10 performs dynamic imaging of a dynamic image of the patient P by directing radiation from the radiation source 12 in a state in which a FPD 13 is disposed at a position facing the radiation source 12 with the patient P interposed therebetween.

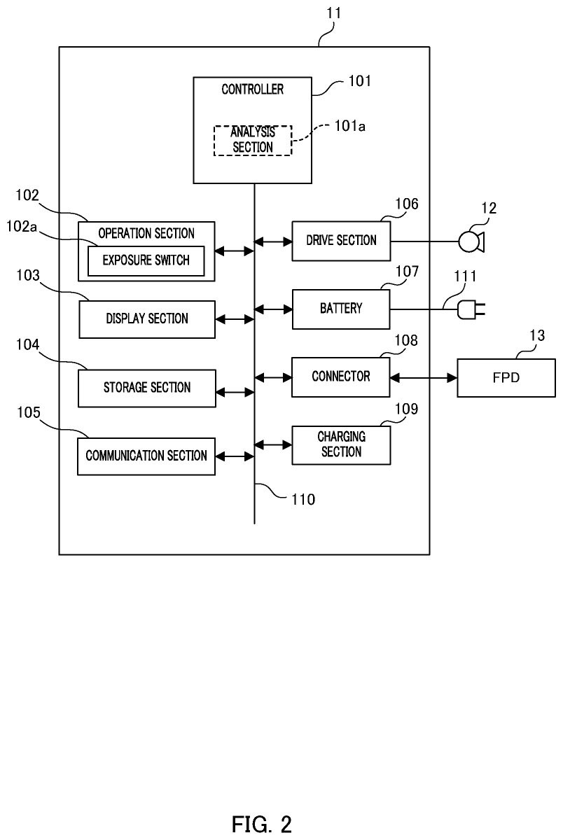

FIG. 2 is a block diagram illustrating the functional configuration of the apparatus main body 11.

The apparatus main body 11 has a function as a console (imaging control apparatus) and also functions as a computer. As shown in FIG. 2, the apparatus main body 11 includes a controller 101, an operation part 102, a display part 103, a storage section 104, a communication section 105, a drive section 106, a battery 107, a connector 108, a charging section 109, and the like. The sections of the apparatus main body 11 are connected to one another by a bus 110.

The controller 101 includes a Central Processing Unit (CPU), a Random Access Memory (RAM), and the like. In response to an input from the operation part 102, the CPU reads out a system program and various processing programs stored in the storage section 104, loads them in the RAM, and executes various processes in accordance with the loaded programs.

The controller 101 controls the dynamic imaging apparatus 10 and may include an analysis section 101a having a function equivalent to that of the dynamic image analysis apparatus 40 to be described later.

The operation part 102 includes a touch screen or the like in which transparent electrodes are arranged in a lattice shape so as to cover the surface of the display part 103. The touch screen detects a position pressed with a finger, a touch pen, or the like, and inputs the position information as operation information to the controller 101. The operation part 102 includes an exposure switch 102a. The exposure switch 102a is used by the user to instruct the radiation source 12 for irradiation and imaging.

The display part 103 includes a monitor such as a Liquid Crystal Display (LCD) or a Cathode Ray Tube (CRT). The display part 103 performs display in accordance with an instruction of a display signal input from the controller 101.

The storage section 104 is composed of a nonvolatile semiconductor memory, a hard disk and the like. The storage section 104 stores various programs executed by the imaging controller 101, parameters necessary for execution of processing by the programs, data such as processing results, and the like.

The communication section 105 performs data transmission and reception to and from the FPD 13 by wired communication or wireless communication, and performs data transmission and reception to and from the dynamic image analysis apparatus 40, the image management apparatus 50, the client terminals 60, and the like connected to the network N via the AP 20.

The drive section 106 is a circuit that drives the radiation source 12. The drive section 106 and the radiation source 12 are connected to each other via a cable.

The battery 107 supplies electric power to each section of the apparatus main body 11 and the radiation source 12. The battery 107 can be charged from the outside via an AC cable 111.

The connector 108 is provided inside the storage section 120 and is electrically connected to the FPD 13 stored in the storage section 120.

The charging section 109 charges the FPD 13 connected via the connector 108 with electric power supplied from the battery 107 under the control of the controller 101.

The dynamic imaging apparatus 10 having the above-described configuration captures dynamic images by irradiating the chest of the patient P during quiet breathing with radiation. Next, as described below, the dynamic image analysis apparatus 40 performs dynamic analysis of the dynamic images and outputs a diagnostic index indicating the respiratory state of the patient P. The diagnostic index output by the dynamic image analysis apparatus 40 may be displayed on the display part 103 of the dynamic imaging apparatus 10 as analysis summaries AS1 and AS2 described below.

### Dynamic Image Analysis Apparatus

The dynamic image analysis apparatus 40 is, for example, a computer such as a PC or a workstation. The dynamic image analysis apparatus 40 may be a desktop computer or a portable computer such as a notebook computer or a tablet computer.

The dynamic image analysis apparatus 40 executes dynamic analysis of the dynamic image captured by the dynamic imaging apparatus 10.

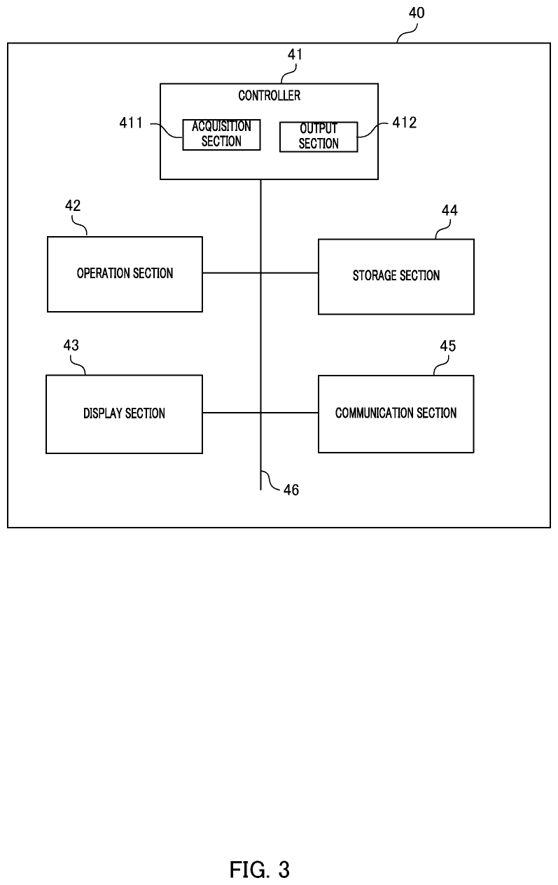

FIG. 3 is a block diagram illustrating an example of a functional configuration of the dynamic image analysis apparatus 40. The dynamic image analysis apparatus 40 includes a controller 41, an operation part 42, a display part 43, a storage section 44, and a communication section 45. The components of the dynamic image analysis apparatus 40 are connected to each other by a bus 36.

The controller 41 is, for example, a computer having one or more hardware processors, and is constituted by a CPU, a RAM, and the like. In the controller 41, in response to an operation on the operation part 42, the CPU reads a system program and various processing programs stored in the storage section 44, loads them in the RAM, and executes operation control of each section of the dynamic image analysis apparatus 40, dynamic analysis, and the like based on the loaded programs. As described below, the controller 41 executes a diagnostic index providing program, an extubation determination information providing program, a respiratory state information providing program, and the like. The diagnostic index providing program, the extubation determination information providing program, the respiratory state information providing program, and the like are stored in a non-transitory computer-readable storage medium, and are stored in the storage section 44 from the storage medium.

The controller 41 includes an acquisition section 411 and an output section 412.

The acquisition section 411 performs the dynamic analysis set in setting information on the dynamic image and acquires an analysis result. For example, the acquisition section 411 performs the dynamic analysis based on a signal change in a plurality of frame images.

The acquisition section 411 has, for example, a blood flow analysis mode, a ventilation analysis mode, an adhesion analysis mode, a diaphragm movement amount analysis mode, a shaping-related measurement mode, and the like as the type of dynamic analysis. Each mode will be briefly described below.

The blood flow analysis mode is a mode in which a signal change in the lung field synchronized with the heartbeat is visualized.

The ventilation analysis mode is a mode in which a signal change in a time direction in a specific time-frequency band is extracted and lung tissue behavior during breathing is visualized.

The adhesion analysis mode is a mode for visualizing the degree of adhesion of tissues.

The diaphragm motion amount analysis mode is a mode in which the up/down motion of the diaphragm accompanying breathing is automatically tracked and the amount of motion of the diaphragm in the up-down direction is quantified.

The shaping-related measurement mode is, for example, a mode in which a change in the position of a specified bone in the four limbs or the like is measured and the trajectory of the movement is displayed.

In the present embodiment, the controller 41 executes the diagnostic index providing program and the respiratory state information providing program as processing programs, and the acquisition section 411 and the output section 412 perform the following processing.

Specifically, in the diagnostic index providing program, the acquisition section 411 performs processing for acquiring respiratory information on respiration and diaphragm information on a diaphragm from a dynamic image captured by irradiating the chest of the patient P during quiet breathing with radiation. The acquisition section 411 can acquire the diaphragm information by using the above-described diaphragm movement amount analysis mode, and can collectively acquire the respiratory information and the diaphragm information by the dynamic analysis of the dynamic image. The output section 412 performs a process of obtaining and outputting a diagnostic index indicating a respiratory state based on the acquired respiratory information and diaphragm information.

Furthermore, in the respiratory state information providing program, the acquisition section 411 performs a process of acquiring a plurality of types of information related to breathing from the dynamic image captured by irradiating the chest of the patient P during quiet breathing with radiation. The plurality of types of information related to breathing are, for example, the above-described respiratory information and diaphragm information, and the acquisition section 411 can acquire the diaphragm information by using the above-described diaphragm movement amount analysis mode and can collectively acquire the plurality of types of information by the dynamic analysis of the dynamic image. The output section 412 performs processing for outputting the plurality of types of acquired information as a list. The output section 412 may output the list including the diagnostic index obtained based on the respiratory information and the diaphragm information.

To further explain, the respiratory information that the acquisition section 411 acquires from the dynamic image is the respiratory rate per minute as described later, and this respiratory information is acquired for each of the left lung and the right lung. Further, the diaphragm information acquired from the dynamic image by the acquisition section 411 is a diaphragm displacement amount as will be described later, and this diaphragm information is also acquired for each of the left lung and the right lung.

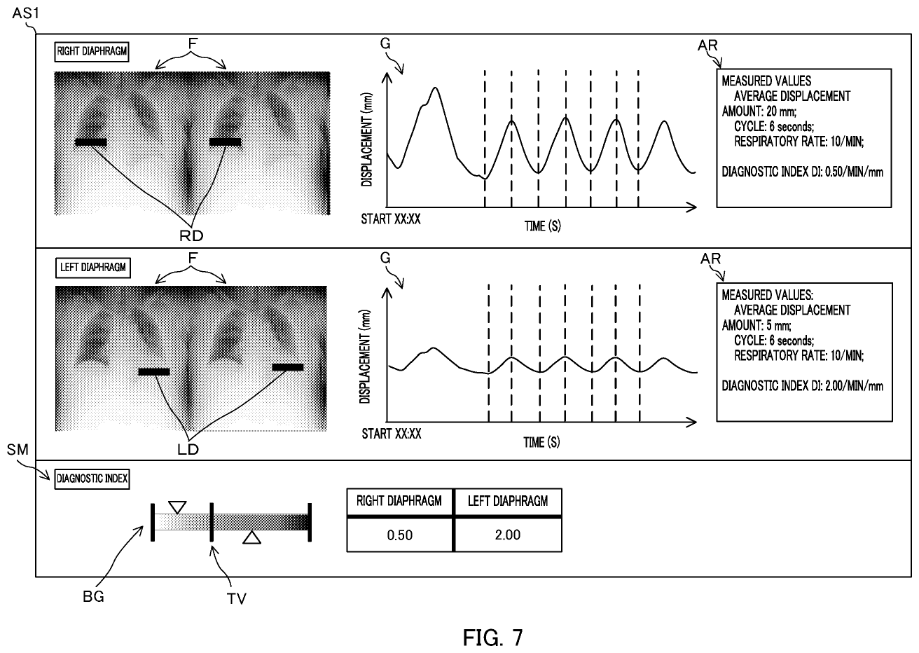

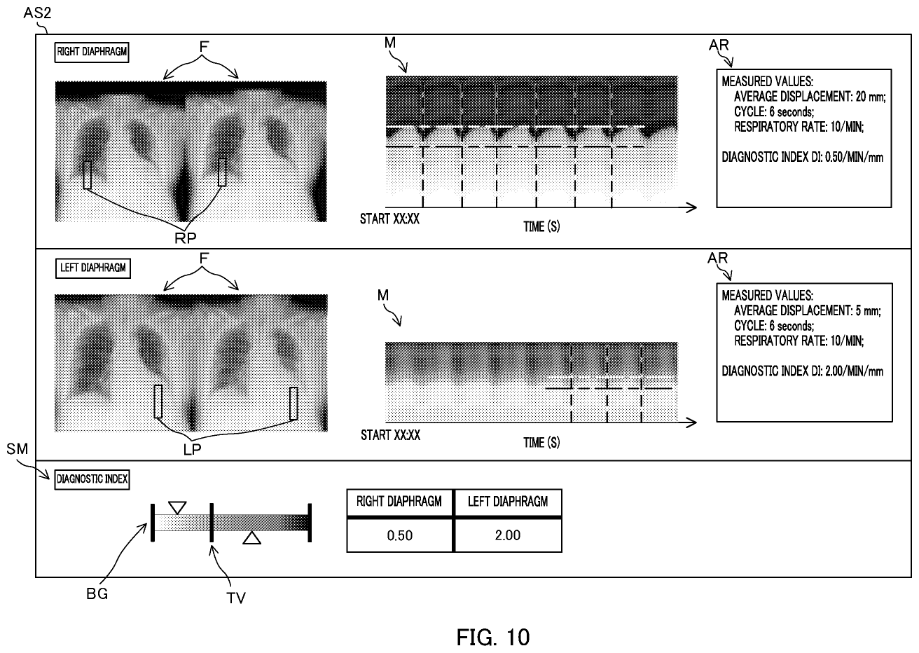

As will be described later, the diagnostic index output by the output section 412 is a value obtained by dividing the respiratory rate by the diaphragm displacement amount, and this diagnostic index is obtained for each of the left lung and the right lung. Next, as illustrated in FIGS. 7 and 10 to be described later, the output section 412 outputs the diagnostic indices together with the dynamic images as the analysis summaries AS1 and AS2 in one screen displayed by the display part 43.

The operation part 42 is an operation device such as a keyboard including cursor keys, number input keys, and various function keys, a pointing device such as a mouse or a trackball, and a touch screen. The operation part 42 generates an instruction signal based on an input of an operator, and outputs the instruction signal to the controller 41. Furthermore, the operation part 42 may include a touch screen on the display screen of the display part 43, and in this case, outputs the instruction signal input through the touch screen to the controller 41.

The display part 43 is constituted by a display device such as a CRT, a liquid crystal display, or an organic EL display. The display part 43 displays the input instruction from the operation part 42, data (the dynamic image, the analysis result, and the like) generated by the dynamic imaging apparatus 10 or the dynamic image analysis apparatus 40, and the like in accordance with an instruction of a display signal input from the controller 41.

The storage section 44 is composed of a nonvolatile semiconductor memory, a hard disk and the like. The storage section 44 stores various programs executed by the controller 41, parameters necessary for execution of processing by the programs, or data such as processing results (dynamic images, analysis results, and the like). The various programs are stored in the form of readable program codes, and the controller 41 sequentially executes operations in accordance with the program codes.

The communication section 45 transmits and receives data to and from the dynamic imaging apparatus 10, the image management apparatus 50, the client terminal 60, and the like.

### Ventilator Weaning Protocol

The ventilator weaning protocol for removing the tube 31 of the ventilator 30 from the patient P includes protocols related to Spontaneous Awakening Trial (SAT) and Spontaneous Breathing Trial (SBT). The protocol related to SAT includes determination of SAT initiation safety criteria, performance of SAT, and determination of SAT success criteria. The protocol related to SBT includes determination of SBT initiation safety criteria, performance of SBT, and determination of SBT success criteria.

The determination of the SAT initiation safety criteria determines whether the administration of a sedative to the patient P may be stopped or reduced in dose. When it is determined that the SAT initiation safety criteria are satisfied, the SAT is performed.

The SAT evaluates whether awakening of the patient P is obtained in a state where administration of a sedative to the patient P is stopped or reduced in dose. After performing SAT, if it is determined that the SAT success criteria are satisfied, the protocol related to SBT is initiated.

Since the dynamic image analysis apparatus 40 having the above-described configuration is used in SBT, detailed description of the SAT initiation safety criteria, the SAT, and the SAT success criteria will be omitted, but known SAT initiation safety criteria, SAT, and SAT success criteria may be used.

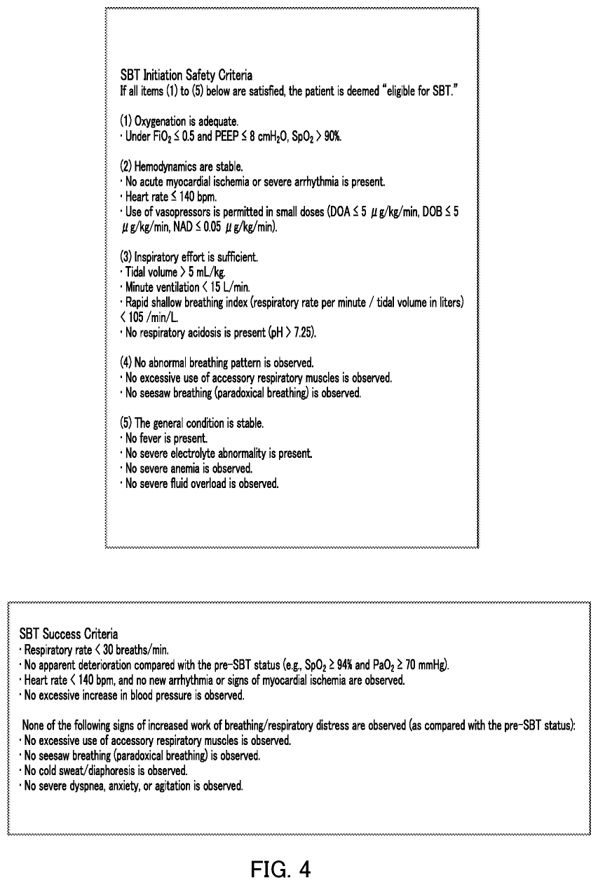

The determination of the SBT initiation safety criteria determines whether or not a transition to a state without assistance to the patient P by the ventilator 30 is allowed. If it is determined that the SBT starting safety criteria illustrated in FIG. 4 are satisfied, SBT is performed.

The SBT evaluates spontaneous breathing of the patient P without assistance to the patient P by the ventilator 30. When it is determined that the SBT success criteria shown in FIG. 4 are satisfied after the SBT is performed, the doctor considers removal of the tube 31 of the ventilator 30.

Here, as illustrated in FIG. 4, main items of the SBT initiation safety criteria include the following (A1) to (A5).
(A1) Oxygenation is sufficient.
(A2) Hemodynamics are stable.
(A3) Inspiratory effort is sufficient.
(A4) No abnormal respiratory pattern is observed.
(A5) General condition is stable.

The main items of the above-described (A1) to (A5) include various sub-items, and detailed description thereof is omitted herein. Among the main items of the above-described (A1) to (A5), “(A3) Inspiratory effort is sufficient.” includes Rapid Shallow Breathing Index (RSBI) as a sub-item thereof, but as described above, there is a report that RSBI has low accuracy as an index for determining extubation. There is also an index called D-RSBI instead of RSBI, but this also has poor reproducibility of measurement as described above.

Therefore, in the present embodiment, instead of the RSBI or the D-RSBI, the dynamic image analysis apparatus 40 is configured to provide a diagnostic index described below as an index that can objectively diagnose the respiratory state during quiet breathing and has high accuracy and reproducibility.

### Provision of Diagnostic Index

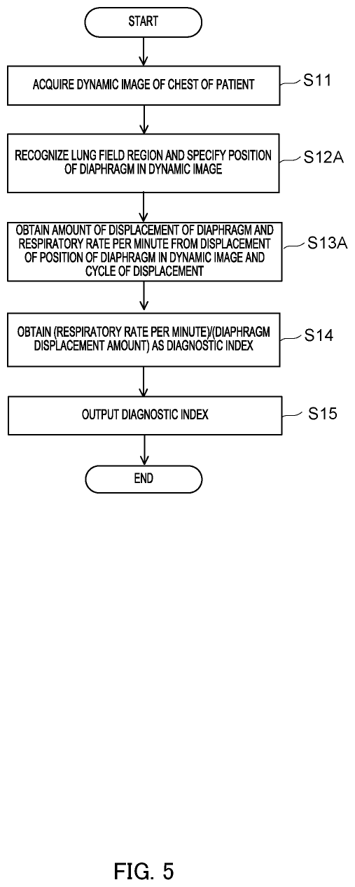

The diagnostic index provided by the dynamic image analysis apparatus 40 will be described below with reference to FIGS. 5 to 7.

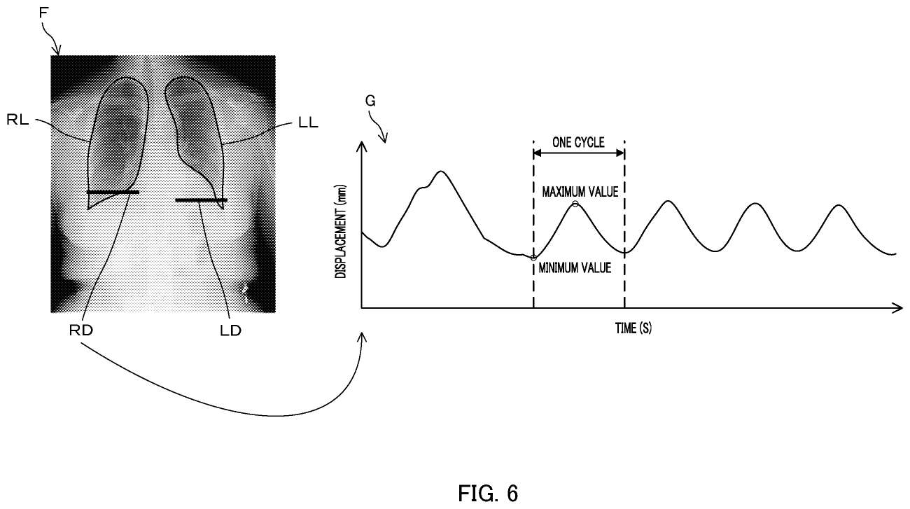

FIG. 5 is a flowchart illustrating a diagnostic index providing method performed by the dynamic image analysis apparatus 40. FIG. 6 is a diagram illustrating a method of obtaining the respiratory rate per minute and a diaphragm displacement amount from dynamic images captured by the dynamic imaging apparatus 10.

As described above, when it is determined that the SAT success criteria are satisfied, the protocol related to the SBT is initiated, and first, the determination of the SBT initiation safety criteria is performed. When the determination of the SBT initiation safety criteria is performed, in the present embodiment, in order to objectively determine the respiratory effort, dynamic imaging of the patient P during quiet breathing is performed by the dynamic imaging apparatus 10. The dynamic image analysis apparatus 40 then outputs a diagnostic index indicating the respiratory state of the patient P during quiet breathing, by a method described below.

### (Step S11)

The controller 41 of the dynamic image analysis apparatus 40 acquires dynamic images of the chest of the patient P obtained by dynamic imaging. The controller 41 acquires, for example, the dynamic images captured by the dynamic imaging apparatus 10 and stored in the storage section 104 via the network N.

### (Step S12A)

The acquisition section 411 of the controller 41 recognizes the lung field region and specifies the position of the diaphragm in each of the plurality of frame images constituting the dynamic image. The lung field region may be recognized, for example, using known image processing such as edge detection or using machine learning or the like. As for the diaphragm, for example, an edge portion on the lower side of the recognized lung field region may be specified as the diaphragm.

For example, as shown in a frame image F in FIG. 6, the acquisition section 411 recognizes lung field regions LL and RL of the right and left lungs, and specifies edge portions on the lower sides of the lung field regions LL and RL as right and left diaphragms LD and RD. Note that the position of the left diaphragm LD is specified in consideration of the position of the heart.

The position information on the diaphragms LD and RD is stored in the storage section 44 for each frame image, and the positions of the diaphragms LD and RD are also displayed when the frame image F is displayed in the analysis summary AS1 illustrated in FIG. 7. Similarly to the diaphragms LD and RD, the position information of the lung regions LL and RL may be stored in the storage section 44 for each frame image, and the positions of the lung regions LL and RL may be displayed when the frame image F is displayed in the analysis summary AS1 shown in FIG. 7.

### (Step S13A)

The acquisition section 411 obtains the respiratory rate per minute and the amount of displacement of the diaphragm from the displacement of the position of the diaphragm in the dynamic image and the cycle of the displacement. The respiratory rate per minute is an example of respiratory information related to respiration, and the displacement amount of the diaphragm is an example of diaphragm information related to the diaphragm.

For example, as illustrated in a graph G in FIG. 6, the acquisition section 411 creates a graph of the displacement of the position of the diaphragm with respect to time (the frame number of the frame image), and obtains the respiratory rate per minute and the displacement amount of the diaphragm from the created graph. Although the graph G in FIG. 6 illustrates the right diaphragm, the left diaphragm is also graphed in the same manner, and the respiratory rate per minute and the amount of displacement of the diaphragm are obtained from the created graph.

For example, the acquisition section 411 detects the cycle of the displacement in the graph G, obtains the respiratory rate per minute from the detected cycle of the displacement, and obtains the displacement amount of the diaphragm from the minimum value and the maximum value of the displacement in one cycle. In a case where a plurality of cycles can be detected, the acquisition section 411 may obtain the respiratory rate per minute from the plurality of cycles, and may obtain an average displacement amount from the displacement amount of the diaphragm in each cycle.

For example, the acquisition section 411 may calculate the similarity of the waveforms of the graph G and set a section to be analyzed so as to include continuous waveforms having a high similarity, or may enable the user to set a section to be analyzed using the operation part 42. In this case, the respiratory rate per minute and the amount of displacement of the diaphragm are obtained in the set section.

### (Step S14, S15)

The output section 412 of the controller 41 obtains a diagnostic index from the respiratory rate per minute and the amount of displacement of the diaphragm acquired by the acquisition section 411 by dividing the respiratory rate per minute by the amount of displacement of the diaphragm, and outputs the obtained diagnostic index. The output diagnostic indices are stored in the storage section 44 together with the acquired respiratory rate per minute and the amount of displacement of the diaphragm, and are displayed as an analysis result in the analysis summary AS1 shown in FIG. 7.

The respiratory rate per minute and the displacement amount of the diaphragm are obtained from a dynamic image in which the entire chest is imaged using radiation, and thus measurement values are not changed by a position, an angle, or the like at which a probe is applied, in contrast to an ultrasonic diagnostic apparatus, and numerical values with high reproducibility can be obtained. Since the respiratory rate per minute and the amount of displacement of the diaphragm are obtained by dynamically analyzing the dynamic image, recognizing the right and left lung fields, and specifying the right and left diaphragms, highly accurate numerical values can also be obtained. As a result, the diagnostic index obtained by the above-described calculation becomes a diagnostic index with high accuracy and reproducibility, and a doctor can objectively diagnose the respiratory state of the patient P during quiet breathing.

FIG. 7 is a diagram illustrating the analysis summary AS1 including the respiratory rate per minute, the diaphragm displacement amount, and the diagnostic index DI obtained by the method described in FIG. 6.

The output section 412 displays, for example, the analysis summary AS1 illustrated in FIG. 7 on the display part 103. The analysis summary AS1 includes the frame images F of the right and left diaphragms, the graph G, the analysis results AR, and the summary SM.

The output section 412 displays, as the frame image F, for example, a frame image when the displacement is the minimum value and a frame image when the displacement is the maximum value. At this time, the output section 412 also displays the diaphragms LD and RD specified in the displayed frame image. Thus, the user can visually recognize the positions of the diaphragms LD and RD in the frame image. Note that the output section 412 may display a dynamic image instead of the frame image F, and even in such a case, it is sufficient to display the diaphragms LD and RD.

The output section 412 displays the graph G and also displays, for example, broken lines indicating the times of the local maximum points and the local minimum points. Thus, the user can visually grasp the cycle and the displacement amount in the graph G.

In addition, the output section 412 displays, as the analysis result AR, the average displacement amount of the diaphragm, the cycle of displacement, the respiratory rate per minute, and the diagnostic index DI obtained by the above-described calculation as numerical values. Thus, the user can objectively grasp the analysis result by the numerical values.

In addition, the output section 412 displays the numerical values of the diagnostic index DI in the right and left diaphragms and a bar graph BG as the summary SM. The bar graph BG is a graph which visually displays the diagnostic index DI, and here, the diagnostic index DI of the right diaphragm is displayed on the upper side of the bar graph BG, and the diagnostic index DI of the left diaphragm is displayed on the lower side of the bar graph BG. In the bar graph BG, the left side of the threshold value TV is the allowable numerical range, and the right side of the threshold value TV is the unallowable numerical range.

In this way, since the output section 412 displays the analysis summary AS1 in one screen of the display part 43, the doctor can check various information including the diagnostic indices DI in the right and left diaphragms and objectively diagnose the respiratory state of the patient P during quiet breathing.

Here, the dynamic imaging apparatus 10 and the dynamic image analysis apparatus 40 are used to determine the SBT initiation safety criteria, but the respiratory state of the patient P may be diagnosed using the dynamic imaging apparatus 10 and the dynamic image analysis apparatus 40 before and after the protocol relating to the SBT. In this case, the setting of the ventilator 30 may be changed based on the respiratory state of the patient P, for example, the diagnostic index DI.

Here, the dynamic image analysis apparatus 40 acquires the diagnostic indices DI from the dynamic images acquired from the dynamic imaging apparatus 10, but the dynamic imaging apparatus 10 may acquire the diagnostic indices DI when the dynamic imaging apparatus 10 includes the analysis section 101a described above. For example, the analysis section 101a may obtain the frame images constituting the dynamic image in real time during the capturing of the dynamic images and obtain the diagnostic index DI for each respiratory cycle. In this case, the dynamic imaging apparatus 10 displays, for example, the analysis summary AS1 including the diagnostic index DI on the display part 103.

### Summary

As described above, the controller 41 of the dynamic image analysis apparatus 40 according to the present embodiment includes the acquisition section 411 and the output section 412 described above.

According to the dynamic image analysis apparatus 40 of the present embodiment, since the diagnostic index DI based on the respiratory information (the respiratory rate per minute) and the diaphragm information (the displacement amount of the diaphragm) has high accuracy and reproducibility as described above, it is possible to provide a diagnostic index having high accuracy and reproducibility. With such a diagnostic index DI, a doctor can objectively diagnose the respiratory state of the patient P during quiet breathing, and when the doctor considers removal of the tube 31 of the ventilator 30, the decision making of extubation can be assisted. In addition, in comparison with the related art, it is possible to easily determine whether or not removal of the tube is eligible, and it is also possible to reduce the psychological burden on the doctor.

In addition, according to the dynamic image analysis apparatus 40 of the present embodiment, a plurality of types of information related to breathing, for example, respiratory information (the respiratory rate per minute), diaphragm information (the amount of displacement of the diaphragm), and the diagnostic index DI are output as a list. Accordingly, it is possible to provide a list of information useful for diagnosis of the respiratory state of the patient P during quiet breathing, and it is possible for a doctor to objectively diagnose the respiratory state of the patient P, and the decision making of extubation can be assisted when the doctor considers extubation of the tube 31 of the ventilator 30. In addition, in comparison with the related art, it is possible to easily determine whether or not removal of the tube is eligible, and it is also possible to reduce the psychological burden on the doctor.

Further, the patient P to whom the ventilator 30 is attached (for example, the patient P in the ICU) is a patient who is difficult to move. It is difficult to move such a patient P to an X-ray imaging room, for example, a Computed Tomography (CT) room, CT imaging cannot be performed, and a high-precision captured image cannot be obtained. Therefore, currently, a method such as an ultrasonic diagnostic apparatus or visual observation by a doctor has to be used for extubation determination, and there has been no means for visualizing the respiratory state in an image including the entire lung. In the present embodiment, the dynamic imaging apparatus 10 can be brought near the patient P by a medical cart on which the dynamic imaging apparatus 10 is mounted. Therefore, according to the dynamic image processing system 1 of the present embodiment, it is possible to acquire a dynamic image including the entire lung without moving the patient P, and it is possible to acquire a diagnostic index indicating a respiratory state.

### Modification Example 1

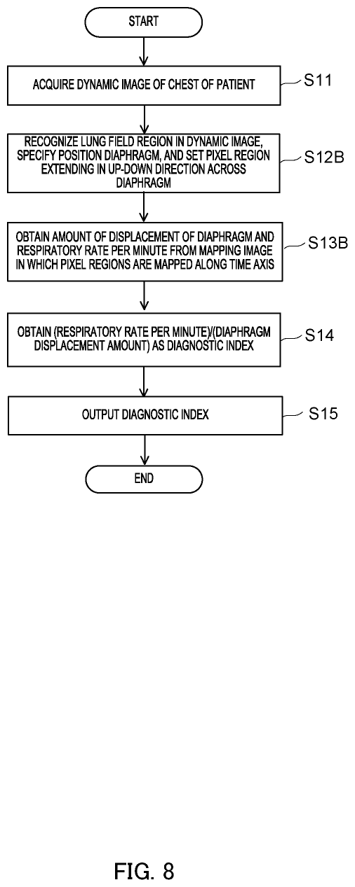

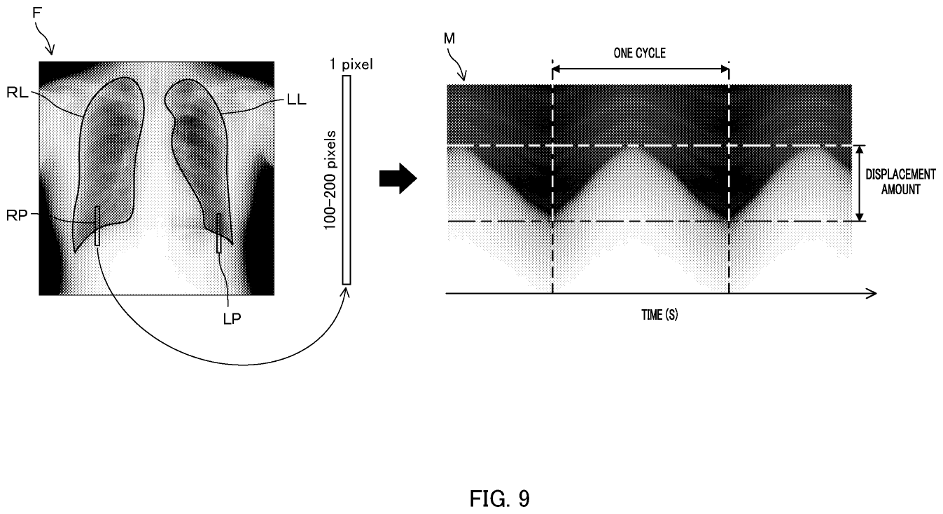

FIG. 8 is a flowchart illustrating a diagnostic index providing method performed by the dynamic image analysis apparatus 40. FIG. 9 is a diagram illustrating another method of obtaining the respiratory rate per minute and a diaphragm displacement amount from dynamic images captured by the dynamic imaging apparatus 10.

Also in the present modification example, the dynamic imaging apparatus 10 and the dynamic image analysis apparatus 40 described in the above embodiment are used, but the diagnostic index providing method performed by the dynamic image analysis apparatus 40 is different from that in the above embodiment. Specifically, the dynamic analysis method for the captured dynamic image is different, and it is described below with reference to FIG. 8 and FIG. 9.

Also in the present modification example, as described above, when the determination of the SBT initiation safety criteria is performed, the dynamic imaging of the patient P during quiet breathing is performed by the dynamic imaging apparatus 10 in order to objectively determine the respiratory effort. The dynamic image analysis apparatus 40 then outputs a diagnostic index indicating the respiratory state of the patient P during quiet breathing, by a method described below.

### (Step S11)

Since this step S11 is the same as step S11 described with reference to FIG. 5, the same reference numerals are given and redundant description is omitted.

### (Step S12B)

The acquisition section 411 of the controller 41 recognizes the left and right lung field regions LL and RL in each of the plurality of frame images constituting the dynamic image, specifies the positions of the left and right diaphragms, and sets the left and right pixel regions LP and RP extending in the up-down direction of the human body across the diaphragm. The recognition of the lung field regions LL and RL and the specification of the position of the diaphragm may be performed by the method described in step S12A of FIG. 5. The pixel regions LP and RP each have a size of, for example, one pixel in the right-left direction of the human body and 100 to 200 pixels in the top-bottom direction of the human body. The size of the pixel regions LP and RP in the up-down direction is set so as to always include the diaphragm even if the diaphragm is displaced.

For example, as shown in a frame image F in FIG. 9, the acquisition section 411 recognizes lung field regions LL and RL, specifies edge portions on the lower sides of the lung field regions LL and RL as right and left diaphragms (reference numerals are omitted), and sets pixel regions LP and RP extending in the up-down direction of the human body across the diaphragm.

The position information on the pixel regions LP and RP is stored in the storage section 44, and the positions of the pixel regions LP and RP are also displayed when the frame image F is displayed in the analysis summary AS2 illustrated in FIG. 10. The position information of the lung regions LL and RL may be stored in the storage section 44 for each frame image, and the positions of the lung regions LL and RL may be displayed when the frame image F is displayed in the analysis summary AS2 shown in FIG. 10.

### (Step S13B)

The acquisition section 411 obtains the amount of displacement of the diaphragm and the respiratory rate per minute from the mapping image M in which the pixel regions LP and RP are mapped along the time axis.

For example, as shown in the mapping image M in FIG. 9, the acquisition section 411 two dimensionally arranges the pixel values in the pixel region RP of each frame image along the time axis (the frame number of the frame image) to form the mapping image M. Then, for example, the acquisition section 411 performs image analysis on the mapping image M, detects displacement of a grayscale boundary (edge) of pixel values with respect to the time axis, that is, displacement of the position of the diaphragm along the time axis, and obtains the respiratory rate per minute and the amount of displacement of the diaphragm from the displacement of the grayscale boundary with respect to the time axis. Note that the mapping image M in FIG. 9 illustrates the right diaphragm, but the left diaphragm is similarly mapped, and the respiratory rate per minute and the amount of displacement of the diaphragm are obtained from the created mapping image.

For example, the acquisition section 411 detects the cycle of the displacement in the mapping image M, obtains the respiratory rate per minute from the detected cycle of the displacement, and obtains the displacement amount of the diaphragm from the minimum value and the maximum value of the displacement in one cycle. In a case where a plurality of cycles can be detected, the acquisition section 411 may obtain the respiratory rate per minute from the plurality of cycles, and may obtain an average displacement amount from the displacement amount of the diaphragm in each cycle.

For example, the acquisition section 411 may calculate the similarity of the waveforms of the detected edges and set a section to be analyzed so as to include continuous waveforms having a high similarity, or may enable the user to set a section to be analyzed using the operation part 42. In this case, the respiratory rate per minute and the amount of displacement of the diaphragm are obtained in the set section.

In the dynamic image, for example, when the contour of the diaphragm is unclear due to a reason such as low contrast of the image, the grayscale boundary of the pixel values, that is, the position of the diaphragm cannot be specified, and it may be difficult to automatically track the position of the diaphragm between the frame images. In this case, in step S12B, the acquisition section 411 sets the pixel regions LP and RP in the left-right direction of the human body to a plurality of pixels instead of one pixel, and averages the pixel values of the plurality of pixels in the left-right direction of the human body. Then, in step S13B, the acquisition section 411 maps, along the time axis, the pixel regions LP and RP obtained by averaging the pixel values of a plurality of pixels in the left-right direction of the human body, to create the mapping image M. Accordingly, even when the contour of the diaphragm is unclear in the dynamic image, the contrast resolution in the up-down direction of the human body can be improved to specify the position of the diaphragm, and the position of the diaphragm can be automatically tracked between the frame images.

### (Step S14, S15)

Since this steps S14 and S15 are the same as the steps S14 and S15 described with reference to FIG. 5, the same reference numerals are given and redundant description is omitted.

Also in the present modification example, the respiratory rate per minute and the displacement amount of the diaphragm are obtained from a dynamic image in which the entire chest is imaged using radiation, and thus measurement values are not changed by a position, an angle, or the like at which a probe is applied, in contrast to an ultrasonic diagnostic apparatus, and numerical values with high reproducibility can be obtained. Since the respiratory rate per minute and the amount of displacement of the diaphragm are obtained by dynamically analyzing the dynamic image, recognizing the right and left lung fields, and specifying the right and left diaphragms, highly accurate numerical values can also be obtained. As a result, the diagnostic index obtained by the above-described calculation becomes a diagnostic index with high accuracy and reproducibility, and a doctor can objectively diagnose the respiratory state of the patient P during quiet breathing.

FIG. 10 is a diagram illustrating the analysis summary AS2 including the respiratory rate per minute, the diaphragm displacement amount, and the diagnostic index DI obtained by the method described in FIG. 9.

The output section 412 displays, for example, the analysis summary AS2 illustrated in FIG. 10 on the display part 43. The analysis summary AS2 includes the frame images F of the right and left diaphragms, the mapping image M, the analysis results AR, and the summary SM.

The output section 412 displays, as the frame image F, for example, a frame image when the displacement is the minimum value and a frame image when the displacement is the maximum value. At this time, the output section 412 also displays the pixel regions LP and RP set in the displayed frame image. Thus, the user can visually grasp the position of the diaphragm and the pixel regions LP and RP in the frame image. Note that the output section 412 may display a dynamic image instead of the frame image F, and even in such a case, it is sufficient to display the pixel regions LP and RP.

In addition, the output section 412 displays the mapping image M and displays, for example, a broken line indicating the time of the local minimum point and an alternate long and short dash line indicating the maximum value and the minimum value of the displacement. Thus, the user can visually grasp the cycle and the displacement amount in the mapping image M.

In the analysis summary AS2, the analysis result AR and the summary SM are the same as those of the analysis summary AS1 illustrated in FIG. 7, and thus the redundant description will be omitted.

In this way, since the output section 412 displays the analysis summary AS2 in one screen of the display part 43, the doctor can check various information including the diagnostic indices DI in the right and left diaphragms and objectively diagnose the respiratory state of the patient P during quiet breathing.

Therefore, also in the present modification example, according to the dynamic image analysis apparatus 40, as described above, it is possible to provide the diagnostic index DI having high accuracy and reproducibility. With such a diagnostic index DI, a doctor can objectively diagnose the respiratory state of the patient P during quiet breathing, and when the doctor considers removal of the tube 31 of the ventilator 30, the decision making of extubation can be assisted. In addition, in comparison with the related art, it is possible to easily determine whether or not removal of the tube is eligible, and it is also possible to reduce the psychological burden on the doctor.

### Modification Example 2

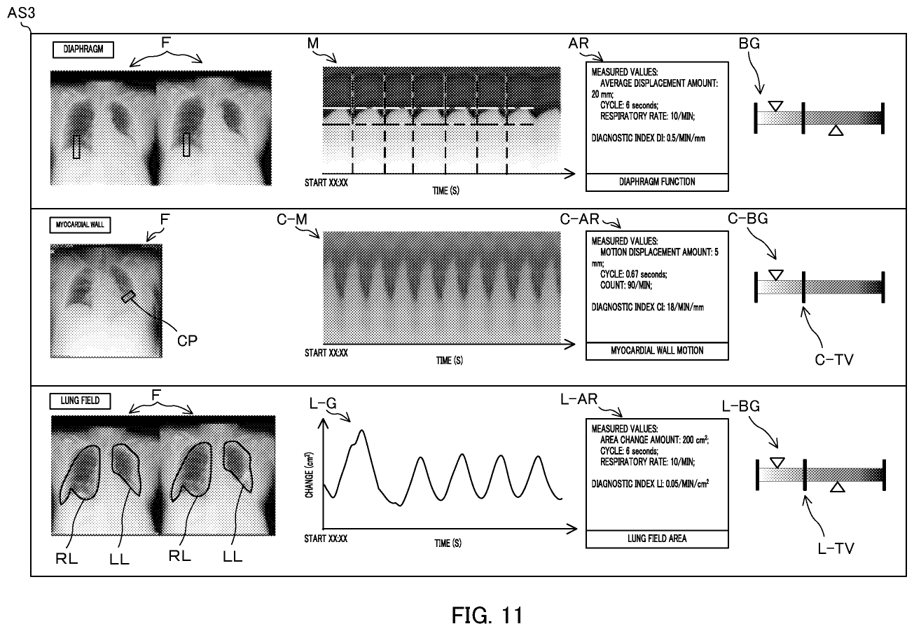

FIG. 11 is a diagram illustrating an analysis summary AS3 including respiratory information and heartbeat information obtained from dynamic images captured by the dynamic imaging apparatus 10.

Although the dynamic imaging apparatus 10 and the dynamic image analysis apparatus 40 described in the above embodiment are used also in the present modification example, a target site on which dynamic analysis is performed by the dynamic image analysis apparatus 40 includes not only the diaphragm but also the heart. The dynamic image analysis apparatus 40 acquires respiratory information and heartbeat information by dynamic analysis of the phrenic and cardiac regions in the dynamic images.

Therefore, in the present modification example, the controller 41 executes an extubation determination information providing program as a processing program, and the acquisition section 411 and the output section 412 perform the following processing.

Specifically, in the extubation determination information providing program, the acquisition section 411 performs a process of acquiring information allowing evaluation of the eligibility of extubation of the ventilator 30 from a dynamic image captured by irradiating the chest with radiation while the patient P to whom the ventilator 30 is attached is in a quiet respiratory state. The information includes respiratory information related to respiration and heartbeat information related to heartbeats, and the acquisition section 411 can collectively acquire the respiratory information and the heartbeat information by a dynamic analysis of a dynamic image, as described later. The output section 412 performs a process of outputting the respiratory information and the heartbeat information as a list.

In addition, in the present modification example, the respiratory information acquired from the dynamic image by the acquisition section 411 includes the respiratory rate, a diaphragm displacement amount, and a respiratory pattern as will be described later, and these pieces of information are acquired for each of the left lung and the right lung. The heartbeat information acquired from the dynamic image by the acquisition section 411 includes a heart rate as will be described later.

In addition, in the present modification example, the respiratory information output by the output section 412 includes the respiratory rate, a diaphragm displacement amount, a respiratory pattern, and a diagnostic index as will be described later, and these pieces of information are obtained for each of the left lung and the right lung. The diagnostic index indicates the respiratory state of the patient P and is also an index with which respiratory effort can be evaluated. The heartbeat information output by the output section 412 includes a heart rate. Then, as illustrated in FIG. 11, the output section 412 outputs a list of information indicating the respiratory rate, the diaphragm displacement amount, the respiratory pattern, the diagnostic indices, and the heart rate together with the dynamic image as the analysis summary AS3 in one screen displayed by the display part 43.

Here, regarding the SBT success criteria illustrated in FIG. 4, main evaluation items to be specifically evaluated are illustrated below.
(B1) Respiratory rate
(B2) Oxygen saturation (SpO₂)
(B3) Partial pressure of carbon dioxide (PaCO₂)
(B4) Heart rate
(B5) Blood pressure
(B6) Respiratory effort
(B7) Respiratory pattern

Of the above-described evaluation items (B1) to (B7), the four evaluation items of; (B1) respiratory rate; (B4) heart rate; (B6) respiratory effort, and (B7) respiratory pattern can be acquired by performing dynamic analysis of the dynamic images with the dynamic image analysis apparatus 40, as described below.

Regarding (B1) respiratory rate, as described above, the dynamic image analysis apparatus 40 can obtain the graph G and the mapping image M indicating the displacement of the B1, and the respiratory rate per minute is obtained from the obtained graph G and mapping image M.

In addition, since the dynamic image analysis apparatus 40 recognizes the lung field regions LL and RL in the frame image F as described above, a change in the lung field area can also be obtained, and the graph L-G showing the change in the lung field area shown in FIG. 11 can also be obtained. The dynamic image analysis apparatus 40 can also obtain the respiratory rate per minute from the obtained graph L-G. In this way, since the dynamic image analysis apparatus 40 obtains the respiratory rate from the displacement of the diaphragm and the change in the lung field area, it is possible to provide the respiratory rate to a doctor as useful information with higher reliability.

Regarding (B6) respiratory effort, as described above, the dynamic image analysis apparatus 40 obtains the diagnostic index DI indicating the respiratory state from the graph G and the mapping image M indicating the displacement of the diaphragm. The diagnostic index DI is an index indicating the respiratory effort of the patient P as the respiratory state.

Furthermore, since the dynamic image analysis apparatus 40 can also obtain the graph L-G as described above, a diagnostic index LI indicating the respiratory state can be obtained from the obtained graph L-G by performing calculation similar to that for the diagnostic index DI. The diagnostic index LI is obtained by, for example, calculation of dividing (the respiratory rate per minute) by (the amount of change in lung field area). The diagnostic index LI is also an index that indicates the respiratory effort of the patient P as a respiratory state.

In the dynamic image analysis apparatus 40, the output section 412 of the controller 41 displays the amount of change in the area of the lung field, the cycle, the respiratory rate per minute, and the diagnostic index LI as numerical values as the analysis result L-AR. Thus, the user can objectively grasp the analysis result by the numerical values.

Further, the output section 412 may display a bar graph L-BG of the diagnostic index LI for the right and left lung fields similarly to the diagnostic index DI. The bar graph L-BG is a graph which visually displays the diagnostic index LI, and here, the diagnostic index LI of the right lung field is displayed on the upper side of the bar graph L-BG, and the diagnostic index LI of the left lung field is displayed on the lower side of the bar graph L-BG. In the bar graph L-BG, the left side of the threshold value L-TV is the allowable numerical range, and the right side of the threshold value L-TV is the unallowable numerical range.

In this way, since the dynamic image analysis apparatus 40 obtains the diagnostic indices DI and LI from the displacement of the diaphragm and the change in the lung field area, it is possible to provide the respiratory effort to a doctor as useful information with higher reliability.

Regarding (B7) respiratory pattern, as described above, the dynamic image analysis apparatus 40 can obtain the graph G indicating the displacement of the diaphragm and the mapping image M. Therefore, the respiratory pattern can be obtained from the graph G and the mapping image M. In the present modification example, the dynamic image analysis apparatus 40 stores a plurality of respiratory patterns indicating respiratory abnormalities in the storage section 44 in advance. Then, the dynamic image analysis apparatus 40 determines whether or not the respiratory pattern from the graph G or the mapping image M is acceptable and the corresponding respiratory abnormality by pattern matching with a plurality of respiratory patterns stored in the storage section 44. Note that the dynamic image analysis apparatus 40 may use, for example, a discriminator that has machine-learned a plurality of respiratory patterns indicating respiratory abnormality to determine whether the respiratory abnormality is acceptable or not and a corresponding respiratory abnormality.

In addition, since the dynamic image analysis apparatus 40 can also obtain the graph L-G as described above, a respiratory pattern can be obtained from the graph L-G. The dynamic image analysis apparatus 40 also determines, with respect to the respiratory pattern obtained from the graph L-G, whether the respiratory pattern is acceptable or not and whether respiratory abnormality occurs therein or not, using pattern matching or the discriminator, as described above. As described above, the dynamic image analysis apparatus 40 determines the respiratory pattern from the displacement of the diaphragm and the change in the lung field area, and thus can provide the doctor with the acceptability of the respiratory pattern and the respiratory abnormality as more reliable and useful information.

(B4) Regarding the heart rate, the dynamic image analysis apparatus 40 applies the above-described diaphragmatic movement amount analysis mode to the myocardial wall, for example, to identify the position of the myocardial wall for each frame image F and create a graph of the displacement of the position of the myocardial wall with respect to time (the frame number of the frame image). Furthermore, as in modification example 1 described above, the dynamic image analysis apparatus 40 may set pixel regions CP that extending across the myocardial wall and create mapping images C-M in which the pixel regions CP are mapped along the time axis. FIG. 11 illustrates a case where the mapping image C-M is created.

The dynamic image analysis apparatus 40 may recognize the lung field region or the heart region by using, for example, edge detection or machine learning, and may specify the position of the myocardial wall or set the pixel region CP based on the recognized lung field region or heart region.

Then, the dynamic image analysis apparatus 40 can obtain the motion displacement amount of the myocardial wall, the cycle of the motion displacement, and the number of motion displacements per minute from the graph of the displacement of the position of the myocardial wall and the mapping image C-M by the same method as the method described in the graph G and the mapping image M described above. As described above, the dynamic image analysis apparatus 40 obtains the heart rate from the graph of the displacement of the position of the myocardial wall and the mapping image C-M, and further obtains the motion displacement amount of the myocardial wall, thus allowing the heart rate to be provided to a doctor as more reliable and useful information.

Note that the dynamic image analysis apparatus 40 may obtain the diagnostic index CI indicating the state of the heart from the motion displacement amount of the myocardial wall, the cycle of the motion displacement, and the number of motion displacements per minute. The diagnostic index CI is obtained by, for example, calculation of dividing (the number of motions per minute) by (the amount of motion displacement of the myocardial wall).

The output section 412 displays, as the analysis result C-AR, the amount of motion displacement of the myocardial wall, the cycle of motion displacement, the number of times of motion displacement per minute, and the diagnostic index CI in numerical values. Thus, the user can objectively grasp the analysis result by the numerical values.

Further, the output section 412 may display a bar graph C-BG of the diagnostic index CI for the heart, similarly to the diagnostic index DI. The bar graph C-BG is a graph that visually displays the diagnostic index CI, and here, the diagnostic index CI for the heart is displayed on the upper side of the bar graph C-BG. In the bar graph C-BG, the left side of a threshold value C-TV is the allowable numerical range, and the right side of the threshold value C-TV is the unallowable numerical range.

As described above, the dynamic image analysis apparatus 40 can collectively acquire, from among the above-described evaluation items (B1) to (B7), the four evaluation items of: (B1) respiratory rate; (B4) heart rate; (B6) respiratory effort; and (B7) respiratory pattern, and provide them as a list. As described above, the (B1) respiratory rate; the (B4) heart rate; the (B6) respiratory effort, and the (B7) respiratory pattern are highly reliable and useful information, and therefore, it is possible to provide the physician with information useful for determining whether to remove the tube from the ventilator collectively in the form of a list. This makes it possible for the doctor to objectively diagnose the respiratory state of the patient P, and when the doctor considers the removal of the tube 31 of the ventilator 30, the decision making of extubation can be assisted. In addition, in comparison with the related art, it is possible to easily determine whether or not removal of the tube is eligible, and it is also possible to reduce the psychological burden on the doctor.

The above-described embodiments are merely examples for implementing the present invention, and the technical scope of the present invention should not be interpreted in a limited manner by these embodiments. That is, the present invention can be implemented in various forms without departing from the spirit or main features thereof.

Although embodiments of the present invention have been described and illustrated in detail, the disclosed embodiments are made for purpose of illustration and example only and not limitation. The scope of the present invention should be interpreted by terms of the appended claims.

What is claimed is:

1. A dynamic image analysis apparatus, comprising:
one or more hardware processors, wherein
the one or more hardware processors are configured to:
collectively acquire respiratory information related to respiration and heartbeat information related to heartbeat, which are information allowing evaluation of eligibility for extubation of a ventilator, from a dynamic image captured by irradiating a chest with radiation while a subject with a ventilator attached thereto is in a quiet breathing state; and
output the respiratory information and the heartbeat information as a list.

2. The dynamic image analysis apparatus according to claim 1, wherein
the one or more hardware processors obtain an index indicating respiratory effort based on the respiratory information and output the list including the index.

3. The dynamic image analysis apparatus according to claim 2, wherein
the respiratory information includes a respiratory rate, a diaphragm displacement amount, and a respiratory pattern.

4. The dynamic image analysis apparatus according to claim 3, wherein
the heartbeat information includes a heart rate.

5. The dynamic image analysis apparatus according to claim 4, wherein
the one or more hardware processors output, as the list, the respiratory rate, the diaphragm displacement amount, the respiratory pattern, and the index for each of left and right lungs.

6. The dynamic image analysis apparatus according to claim 5, further comprising:
a display, wherein
the one or more hardware processors output information indicating the respiratory rate, the diaphragm displacement amount, the respiratory pattern, the index, and the heart rate as the list together with the dynamic image in one screen displayed by the display.

7. A dynamic image processing system, comprising:
a medical cart including a dynamic imaging apparatus for capturing a dynamic image by irradiating a chest with radiation while a subject with a ventilator attached thereto is in a quiet breathing state, and a cart in which the dynamic imaging apparatus is mounted and that is configured to be movable; and
the dynamic image analysis apparatus according to claim 1.

8. A non-transitory computer-readable storage medium storing an extubation determination information providing program for causing a computer of a dynamic image analysis apparatus to execute:
a process of collectively acquiring respiratory information related to respiration and heartbeat information related to heartbeat, which are information allowing evaluation of eligibility for extubation of a ventilator, from a dynamic image captured by irradiating a chest with radiation while a subject with a ventilator attached thereto is in a quiet breathing state; and
a process of outputting the respiratory information and the heartbeat information as a list.
